(19) United States
(12) Patent Application Publication
(10) Pub. No.: US 2009/0264993 A1
(43) Pub. Date: Oct. 22, 2009

(54) Endovascular Prosthesis for Ascending Aorta

(75) Inventor: Trevor Greenan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/105,527

(22) Filed: Apr. 18, 2008

(51) Int. Cl. A61F 2/06

(52) U.S. Cl. 623/1.36

(57) ABSTRACT

An endoluminal prosthesis for use in the ascending aorta is disclosed. The prosthesis includes a tubular graft material having an outer surface and an inner surface, a support structure coupled to the graft material, and a plurality of anchors extending from a proximal end of the support structure. Each anchor is curved outwardly such that a middle portion of the anchor extends further outwardly than a proximal portion of the anchor and a distal portion of the anchor such that the anchors conform to the shape of the aortic root. A hook extends outwardly from a proximal end of each anchor to engage the annulus of the aortic valve. The proximal end of the support structure and graft material is disposed adjacent the sinotubular junction.

### FIELD OF THE INVENTION

The invention relates to a medical device for use within a body vessel and, in particular, to an endovascular prosthesis for use in the ascending aorta.

### BACKGROUND

The aorta is the major artery that carries blood from the heart to the rest of the body. FIG. 1 is a schematic illustration of the aorta 100 and the heart 108. The aorta 100 includes an ascending aorta 102, an aortic arch 104, and a descending aorta 106. The ascending aorta 102 is the first segment of the aorta 100 where the aorta 100 originates from the from the heart's left ventricle. Coronary arteries 110 originate at the aortic root 111. The brachlocephalic artery 116, the left common carotid artery 118, and the left subclavian artery 120 branch from the aortic arch 104. The descending artery 106 extends past the diaphragm 112, leading to the abdominal aorta 114.

Aortic dissection occurs when the inner layer of the aorta's artery wall splits open (dissects). This is more likely to occur where pressure on the artery wall from blood flow is high, such as the ascending aorta 102. FIG. 2 shows a dissection 122 in the ascending aorta 102. The dissection may be caused by a tear 124 in the aortic wall. When the aortic wall splits, the pulses of blood get inside the artery wall and under the inner layer, creating a false lumen 126 in which blood flows. This makes the aorta 100 split further. This split may continue distally away from the heart 108 through the aortic arch 104 and down the descending aorta 106 and into its major branches or it may sometimes run proximally back toward the heart 108.

The ascending aorta 102 and aortic arch 104 may also be affected aneurysmal dilatation. The standard surgical approach in patients with ascending aortic aneurysm or dissection involving the aortic root and associated with aortic valve disease is the replacement of the aortic valve and ascending aorta by means of a composite valve graft onto which are reattached the two coronary arteries 110. If the aortic valve leaflets are normal, a valve-sparing aortic root remodeling procedure which keeps the natural patient valve on site is a reasonable alternative in certain individuals. These open surgical operations rely upon cardiopulmonary bypass, with or without hypothermic circulatory arrest. The associated mortality, morbidity, debility, pain and expense are all high.

Endovascular methods of reconstruction in the ascending aorta and aortic arch face difficulty in finding healthy vessel tissue on which to land an endovascular prosthesis or stent-graft. As shown in FIGS. 3 and 4, common endovascular prostheses or stent-grafts 150 for use in the descending aorta 106 include a graft material 154, such as woven polymer materials (e.g., Dacron (polyester) or polytetrafluoroethylene (“PTFE”)), and a support structure 152. The support structure 152 expands in the vessel to hold the graft 150 against the vessel wall. The stent-grafts typically have graft material secured onto the inner diameter or outer diameter of the support structure that supports the graft material and/or holds it in place against a vessel wall. The prosthesis is typically secured to a vessel wall upstream and downstream of the aneurysm site spanning the aneurysm with at least one attached expandable annular spring member that provides sufficient radial force so that the prosthesis engages the inner vessel wall of the body lumen to seal the prosthetic lumen from the aneurysm. The spring member needs to be positioned to expand, i.e. land, on a healthy portion of the vessel tissue to hold the prosthesis in place. In a dissection or aneurysm in the ascending aorta, there may not be suitable healthy tissue at one or both ends of the dissection or aneurysm on which to land the spring member.

### SUMMARY OF THE INVENTION

An endoluminal prosthesis includes a tubular graft material having an outer surface and an inner surface, a support structure coupled to the graft material, and a plurality of anchors extending from a proximal end of the support structure. Each anchor is curved outwardly such that a middle portion of the anchor extends further outwardly than a proximal portion of the anchor and a distal portion of the anchor. Each anchor further includes a hook extending outwardly from a proximal end the anchor.

In a method for treating a diseased portion of the ascending aorta, the prosthesis is delivered to the ascending aorta such that the proximal end of each anchor is aligned with annulus of the aortic valve, the middle portion of each anchor is disposed adjacent the sinuses, and the distal end of each anchor is disposed adjacent the sinotubular junction. The prosthesis is deployed such that the hooks at the proximal ends of the anchors engage the annulus and the support structure and graft material expand to contact an inner surface of the ascending aorta. Each anchor preferably extends through a junction between adjacent leaflets of the aortic valve.

### BRIEF DESCRIPTION OF DRAWINGS

The foregoing and other features and advantages of embodiments according to the present invention will be apparent from the following description as illustrated in the accompanying drawings. The accompanying drawings, which are incorporated herein and form a part of the specification, further serve to explain the embodiments and to enable a person skilled in the pertinent art to make and use embodiments thereof. The drawings are not to scale.

FIG. 1 is a schematic illustration of the heart and the aorta.

FIG. 2 is a schematic illustration of the aorta with a dissection.

FIGS. 3 and 4 are schematic illustrations of a prior art endoluminal prosthesis for use in treating an aneurysm in the descending aorta.

FIG. 5 is a cross-sectional illustration of the ascending aorta with a dissection and the aortic root.

FIG. 6 is a schematic illustration of the aortic valve.

FIG. 7 is a schematic illustration of an endoluminal prosthesis according to an embodiment hereof.

FIG. 7A is a detailed view of a portion the prosthesis of FIG. 7.

FIG. 8 is a schematic side view of the prosthesis of FIG. 7.

FIG. 9 is a cross-sectional view of the prosthesis of FIG. 7 deployed in the ascending aorta.

FIG. 10 is a schematic view of the aortic valve with the prosthesis of FIG. 7 deployed as in FIG. 8.

FIGS. 11-13 are schematic illustrations of the steps of a method of delivering the prosthesis of FIG. 7 to the ascending aorta and deploying the prosthesis therein.

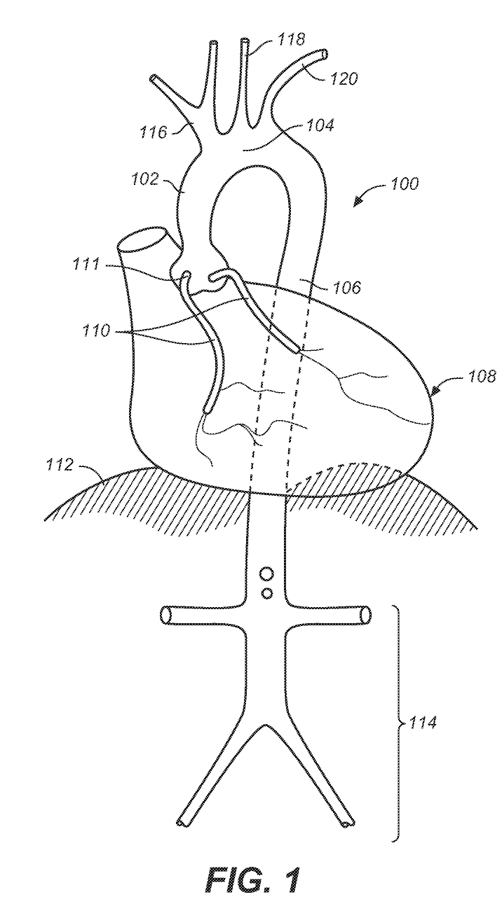

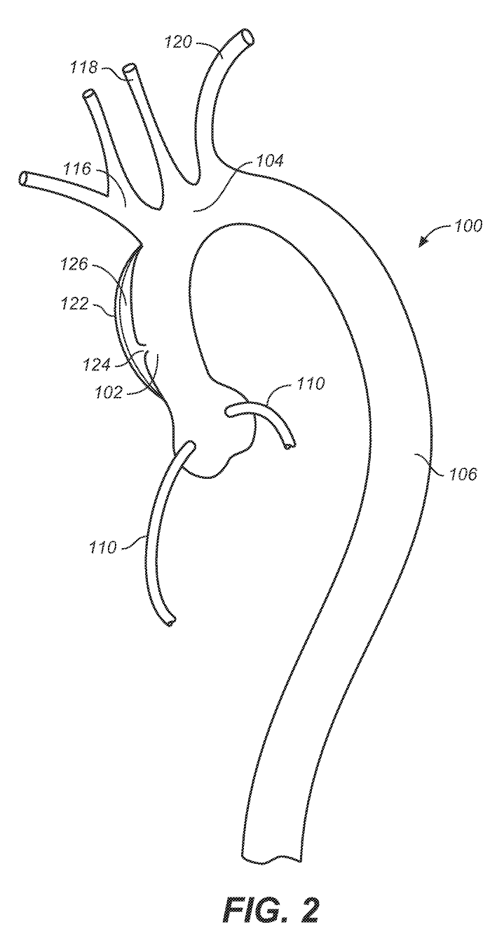

### DETAILED DESCRIPTION

Specific embodiments are now described with reference to the figures, wherein like reference numbers indicate identical or functionally similar elements. The terms “distal” and “proximal” are used in the following description with respect to a position or direction relative to the heart. “Distal” or “distally” are a position distant from or in a direction away from the heart. “Proximal” and “proximally” are a position near or in a direction toward the heart.

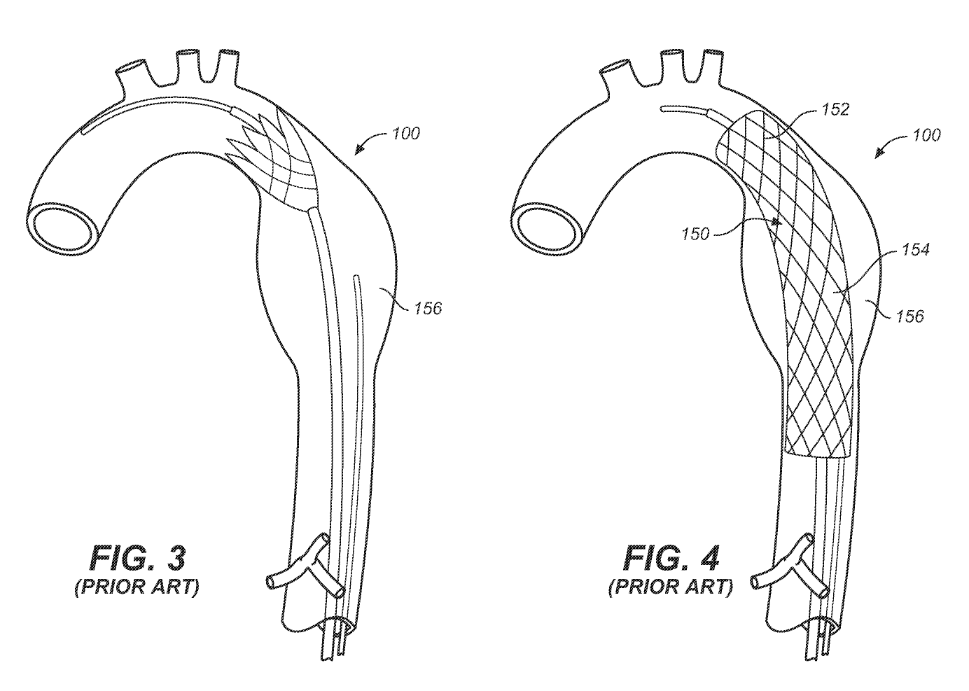

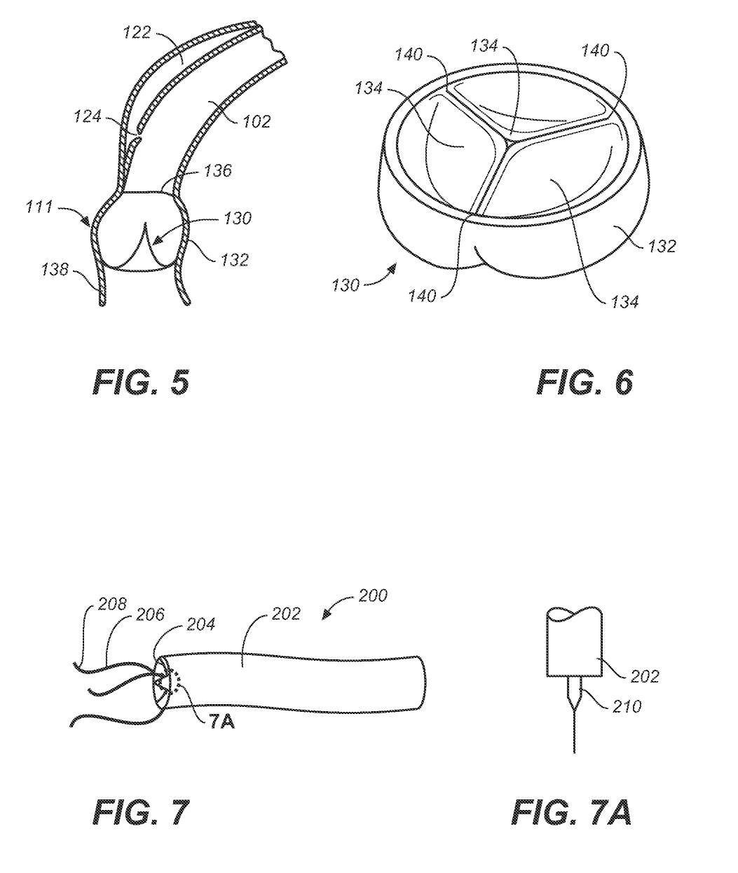

FIG. 5 is a schematic illustration of the junction between the ascending aorta 102 and the heart. The aortic root 111 is the portion of the left ventricular outflow tract which supports the leaflets 134 (shown in FIG. 6) of the aortic valve 130. The aortic root 111 may be delineated by the sinotubular junction 136 distally and the bases of the valve leaflets 134 proximally. The aortic root 111 comprises the sinuses 132, the valve leaflets 134, the commissures 140, and the interleaflet triangles (not shown). The annulus 138 is the area of collagenous condensation at the point of leaflet attachment. The annulus 138 comprises a dense fibrous ring attached either directly or indirectly to the atrial or ventricular muscle fibers. Due to the structure of the annulus 138, it can provide a suitable landing zone for an endovascular graft.

FIG. 7 is a schematic illustration of an endovascular prosthesis 200 in accordance with an embodiment hereof. In this embodiment, prosthesis 200 is a stent-graft including a tubular graft material 202 coupled to a series of radially compressible annular support members 204. The annular support members 204 support the graft and/or bias the prosthesis 200 into conforming fixed engagement with an interior surface of the ascending aorta 102 (see FIG. 9). The annular support members 204 are spring members having predetermined radii and are constructed of a material such as Nitinol in a superelastic, shape set condition. The graft material 202 may be a woven polymer material (e.g., Dacron (polyester) or polytetrafluoroethylene (“PTFE”)) or other suitable graft material known to those of ordinary skill in the art.

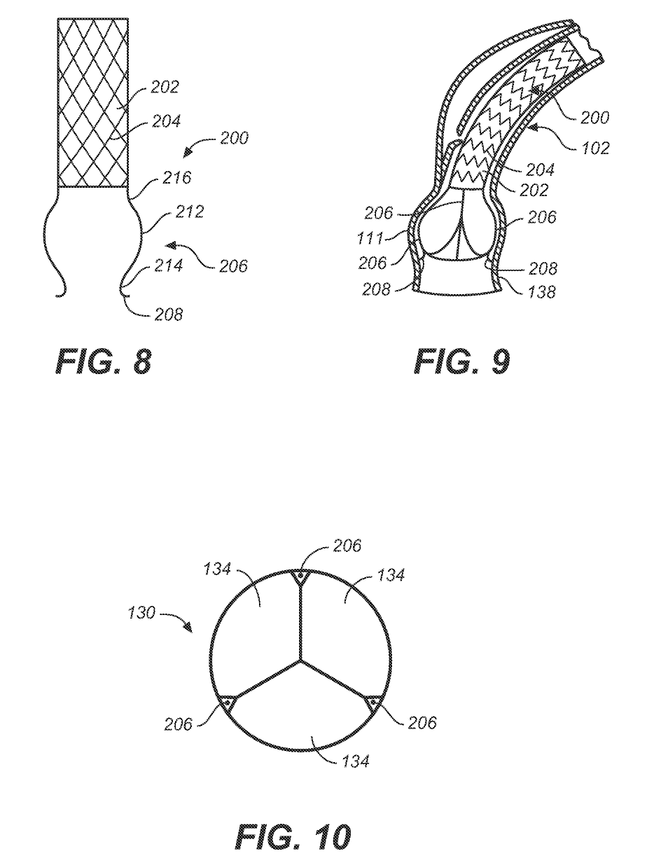

Anchors 206 extend from a proximal end of prosthesis 200. In the embodiment shown in FIG. 7, the prosthesis 200 includes three anchors 206. Anchors 206 are coupled to support members 204 and are preferably formed from the same material as support members 204. Anchors 206 may be welded to support members 204 or may be formed with support members 204, or coupled to support members 204 in any other manner known to those of ordinary skill in the art. Anchors 206 are curved such that a middle portion 212 of each anchor extends further outwardly than a proximal portion 214 and a distal portion 216, as shown in FIG. 8. Anchors 206 are curved to conform to the shape of the sinuses 132 and each anchor 206 includes a hook 208 at its proximal end. Hooks 208 curve outwardly such that hooks 208 can engage the annulus 138. Distal portion 216 of anchors 206 may include a widened portion 210, as shown in FIG. 7A. The widened portion is created by changing the cross section of the anchor (wire connector or strut) at locations where it is likely to be adjacent to more delicate tissue, outside the area near the aortic valve and annulus. While the tissues around the valve are robust and supported by cartilage or cartilage like tissue, other areas of the proximal aorta are fragile and subject to a cheese cutting effect by thin anchor (connecting) wires. To reduce the chance of tissue damage, the cross section of the anchor wire is expanded to create a larger area for the distribution of any contact force between the anchor (connecting strut or element) and the adjacent tissue. This larger are reduces the localized peak force on the tissue and reduces the likelihood that a cheese cutting type damage will occur. Minimizing the cross-sectional area in the commissures limits any impact on valve performance. The increase in cross sectional area to reduce the chance of damage to surrounding tissue can also be created by applying a thick coating to the anchor (connection elements or hook struts) to effectively increase their surface area to reduce the force per unit area imposed on the surrounding tissue. Although three anchors 206 are shown in FIG. 7, one of ordinary skill in the art would understand that more or less anchors could be used. For example, two or four anchors 206 could be used. Three anchors 206 are preferred for a tricuspid valve such that each of the anchors 206 may extend through the junction between adjacent leaflets.

FIG. 9 is a cross-sectional view of the prosthesis 200 installed in the ascending aorta 102. Anchors 206 extend from the annulus 138 to the sinotubular junction 136. Hooks 208 at the proximal ends of anchors 206 engage the annulus 138 to anchor prosthesis 200. Anchors 206 are shaped to conform to the sinuses 132 (FIG. 5) of the aortic root 111. The proximal most support member 204 is located at the sinotubular junction 136 and the remainder of the prosthesis 200 extends distally therefrom. Anchors 206 also serve to support the commissures 140 (FIG. 5) of valve 130. Graft material 202 covers tear 124 such that blood does not flow to false lumen 126.

FIG. 10 is a schematic top view of valve 130 with prosthesis 200 installed. Anchors 206 extend through valve 130 at the junction between adjacent leaflets 134. Anchors 206 therefore do not disturb the operation of valve 130. Further, by engaging annulus 138 just proximal to and adjacent to commissures 140, anchors 206 serve to support commissures 140.

Although endovascular prosthesis 200 of the present embodiment is a stent-graft, endovascular prosthesis 200 may be any prosthetic device for use in a vessel, in particular, the ascending aorta. Further, anchors 206, either alone or in combination with all or a portion of prosthesis 200, may be used as a docking station to which other devices may be coupled.

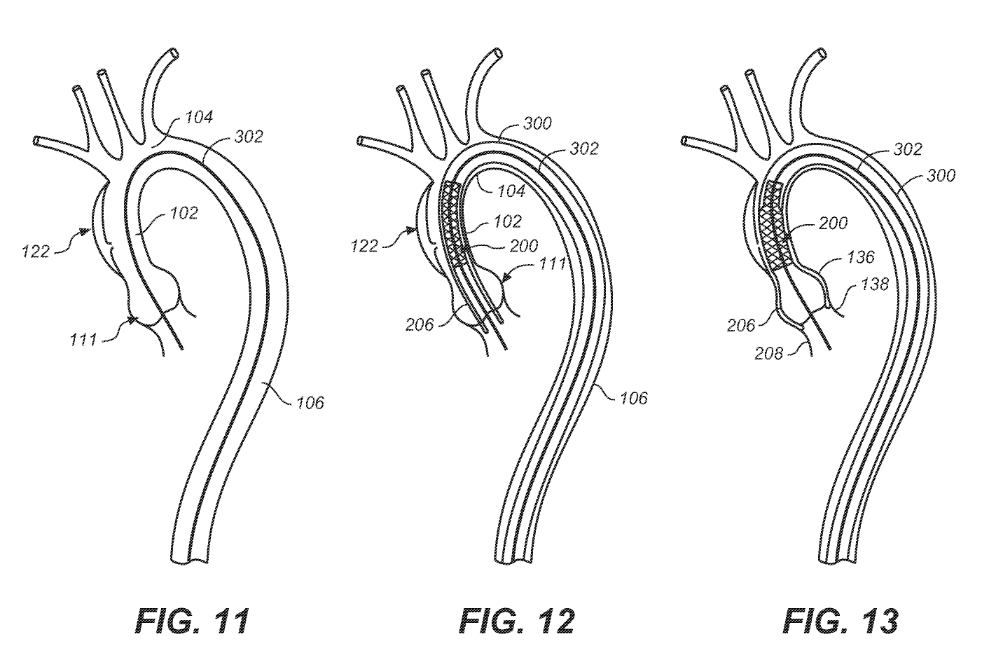

FIGS. 11-13 diagrammatically illustrate delivering prosthesis 200 to the ascending aorta 102 and deploying prosthesis 200. A guidewire 302 is tracked through the descending aorta 106, aortic arch 104, ascending aorta, 102 and aortic valve. Access can be gained through the femoral artery or other locations as would be understood by those of ordinary skill in the art. A catheter 300 with prosthesis 200 enclosed therein is tracked along guidewire 302 to the aortic valve, as shown in FIG. 12. Catheter 300 is shown diagrammatically and may include several components known to those of ordinary skill in the art, such as inner and outer tubes, a sheath, and a pusher. Prosthesis 200 is oriented such that anchors 206 align with the junction of the valve leaflets 134. After catheter 300 is in place at aortic valve, catheter 300 is withdrawn distally (away from the heart) to release prosthesis 200, as shown in FIG. 13. Hooks 208 of anchors 206 engage the annulus 138. Anchors 206 are shaped to conform to sinuses 136. After catheter 200 is completely withdrawn and guidewire 302 is removed, prosthesis 200 is deployed within ascending aorta 102 as shown in FIG. 9.

Although prosthesis 200 has been shown extending only within the ascending aorta 102, it would be understood by those of ordinary skill in the art that the prosthesis may extending into the aortic arch 104 and into the descending aorta 106, if necessary. In such a situation, prosthesis 200 must accommodate branches 116, 118, 120, which can be done by means known to those of ordinary skill in the art. For example, openings may be provided in graft material 202 and prosthesis may be oriented such that the openings align with branches 115, 118, and 120, as explained in U.S. Published Patent Application Publication No. 2007/0233229, which is incorporated by reference herein in its entirety. Alternatively, a modular system may be provided as explained in U.S. Pat. No. 6,814,572, which is incorporated by reference herein in its entirety.

While various embodiments according to the present invention have been described above, it should be understood that they have been presented by way of illustration and example only, and not limitation. It will be apparent to persons skilled in the relevant art that various changes in form and detail can be made therein without departing from the spirit and scope thereof. It will also be understood that each feature of each embodiment discussed herein, and of each reference cited herein, can be used in combination with the features of any other embodiment. All patents and publications discussed herein are incorporated by reference herein in their entirety.

What is claimed is:

1. An endoluminal prosthesis comprising:
a tubular graft material having an outer surface and an inner surface;
a support structure coupled to the graft material; and
a plurality of anchors extending from a proximal end of the support structure, wherein each anchor is curved outwardly such that a middle portion of the anchor extends further outwardly than a proximal portion of the anchor and a distal portion of the anchor.

2. The prosthesis of claim 1, wherein each anchor further includes a hook extending outwardly from a proximal end the anchor.

3. The prosthesis of claim 1, wherein the support structure is coupled to the inner surface of the tubular graft material.

4. The prosthesis of claim 1, wherein the support structure is coupled to the outer surface of the tubular graft material.

5. The prosthesis of claim 1, wherein the prosthesis includes three anchors.

6. The prosthesis of claim 1, wherein the prosthesis includes two anchors.

7. The prosthesis of claim 1, wherein the distal portion of each anchor includes a widened portion.

8. A method for treating a diseased portion of the ascending aorta comprising the steps of:
delivering a prosthesis to the ascending aorta, wherein the prosthesis includes a plurality of anchors having a proximal end, a middle portion, and a distal end;
aligning the prosthesis such that the proximal end of each anchor is disposed adjacent the annulus of the aortic valve, the middle portion of each anchor is disposed adjacent the sinuses, and the distal end of each anchor is disposed adjacent the sinotubular junction;
deploying the prosthesis such that hooks at the proximal ends of the anchors engage the annulus.

9. The method of claim 8, wherein the prosthesis further includes a tubular graft material having an outer surface and an inner surface and a support structure coupled to the graft material, wherein the plurality of anchors are coupled to and extend from a proximal end of the support structure.

10. The method of claim 9, wherein the step of deploying the process further includes the expanding the support structure and the graft material to contact an inner surface of the ascending aorta.

11. The method of claim 8, wherein each anchor is curved such that the middle portion of each anchor extends further outwardly than the proximal end of the anchor and the distal end of the anchor such that the middle portion conforms generally to the shape of the sinuses of the aortic root.

12. The method of claim 8, wherein each anchor extends through a junction between adjacent leaflets of the aortic valve.

13. The method of claim 8, wherein the prosthesis includes three anchors.

14. The method of claim 8, wherein the prosthesis includes two anchors.

15. The method of claim 8, wherein a distal portion of each anchor includes a widened portion.

16. The method of claim 9, wherein the diseased portion of the ascending aorta includes a dissection.

17. The method of claim 9, wherein the diseased portion of the ascending aorta includes an aneurysm.

18. An endoluminal device comprising:
a prosthesis including a plurality of anchors extending longitudinally in a tubular arrangement, wherein each anchor includes a proximal portion, a middle portion, and a distal portion, and wherein each anchor is curved outwardly such that the middle portion of the anchor extends further outwardly than a proximal portion of the anchor and a distal portion of the anchor.

19. The endoluminal device of claim 18, wherein the prosthesis includes a tubular graft material and a support structure coupled to the graft material, wherein the anchors are coupled to and extend proximally from a proximal end of the support structure.

20. The endoluminal device of claim 18, wherein the prosthesis includes a tubular graft material and a support structure coupled to the graft material, wherein the anchors are coupled to and extend proximally from a proximal end of the graft material.

21. The endoluminal device of claim 18, wherein each anchor includes a hook extending outwardly from a proximal end of the anchor.
